(19) United States
(12) Patent Application Publication
(10) Pub. No.: US 2010/0131059 A1
(43) Pub. Date: May 27, 2010

(54) INTRAOCULAR LENS OPTIC

(75) Inventors: Wayne B. Callahan, Abingdon, VA (US); Paul S. Koch, Warwick, RI (US); Anna S. Hayes, Newton Centre, MA (US); Robert E. Kellan, Methuen, MA (US)

Correspondence Address: REMENICK PLLC, 1025 THOMAS JEFFERSON STREET, NW, WASHINGTON, DC, 20007, US

(73) Assignee: Anew Optics, Inc.

(21) Appl. No.: 12/626,459

(22) Filed: Nov. 25, 2009

Related U.S. Application Data
(60) Provisional application No. 61/118,076, filed on Nov. 26, 2008.

(51) Int. Cl. A61F 2/16

(52) U.S. Cl. 623/6.23

(57) ABSTRACT

An intraocular lens optic (e.g. FIG. 1) having a maximum thickness of 500 microns (3) and a diameter of 6 millimeters, with concentric rings on the anterior surface of the lens. The lens, coupled with suitable haptic designs, is to be implanted within the lens capsule (19) of the eye after surgical removal of the natural crystalline lens. The anterior surface of the lens (1) has concentric rings (6) with steps of approximately 10 microns (5) that can be concave, convex or piano, with the edge of the step parallel in each case to the light rays traversing the lens at that point. The posterior surface of the lens (3) is aspherical and smooth. The concentric rings focus 95% or better of light at a specific target point on the retina, thus making a monofocal lens, with focal flexibility provided through haptic design providing movement of the lens forward in the posterior chamber in response to contraction and expansion of the ciliary body and concomitant repositioning of the zonules. The inventive lens is a unitarily formed, seamless body comprised preferably of hydrophilic acrylates or acrylates and silicone blends. Other possible materials include hydrophobic acrylates, polymethylmethacrylate (such as for example PMMA) or acrylic blends. The inventive lens, being less than 500 microns thick, provides greater transfer of light through the lens, thus more closely replicating the function of a natural, emmotropic lens, while the thinness, making the lens lightweight, allows the ciliary body to move the lens with less effort, thus facilitating comfort in the presbyopic eye.

### REFERENCE TO RELATED APPLICATIONS

This application claims priority to U.S. Provisional Application No. 61/118,076 of the same title and filed Nov. 26, 2008, the entirety of which is hereby incorporated by reference.

### BACKGROUND

1. Field of the Invention

This invention is directed to intraocular lenses that provide increased comfort and performance to the patient. In particular, the invention is directed to intraocular lenses that are no more than 500 microns in thickness and can possess concentric rings on the anterior surface, and to methods of forming these lenses.

2. Description of the Background

Many individuals over the age of fifty years suffer opacification of the crystalline lens of the eye; a condition known as cataracts. Cataracts are progressive and can occur in both eyes and result in significant reduction in visual acuity. Patients with cataracts often see starbursts or other blinding glares when confronted with direct, strong beams of light, such as automobile headlamps. Fading vision and possible ultimate blindness due to cataracts can only be corrected by surgically removing the crystalline lens and replacing the lens with an artificial lens. Many patients have received lenses that, while providing improved base vision, still produce halos, rings, rainbows or other blurring, and many current cataract lenses do not provide the focal flexibility to allow the patient to adjust visual distances, specifically from far to near vision, though also in the intermediate ranges, thus requiring eyeglasses or contact lenses in addition to the aphakic cataract lens.

Other individuals that suffer from vision problems, which normally require prescription lenses, either contact lenses or eyeglasses, to correct. These patients suffer from cases of hyperopia, myopia, or presbyopia, and, when given the choice of the aforementioned corrections or surgical alternatives, may elect to have a clear lens replacement in which the natural crystalline lens is removed surgically and an aphakic lens is placed within the lens envelope. An artificial lens can be used for clear lens replacement, providing suitable corrective vision to each affected eye and thereby mitigating the need for other corrective lenses. One example that can provide corrective vision is an aspheric lens. An aspheric lens or asphere is a lens whose surfaces have a profile that is neither a portion of a sphere nor of a circular cylinder. The asphere's more complex surface profile can eliminate spherical aberration and reduce other optical aberrations compared to a simple lens. As such, a single aspheric lens can often replace a much more complex multi-lens system. The resulting lens is smaller and lighter, and possibly less expensive than a multi-lens design.

Like other lenses for vision correction, aspheric lenses can be categorized as convex or concave. Convex aspheric curvatures are used in many presbyopic vari-focal lenses to increase the optical power over part of the lens, aiding in near-pointed tasks such as reading. The reading portion is an aspheric “progressive add.” Also, in aphakia or extreme hyperopia, high plus power aspheric lenses can be prescribed, but this practice is becoming obsolete, replaced by surgical implants of intra-ocular lenses. Many convex types of lens have been approved by governing agencies regulating prescriptions.

Concave aspheres are used for the correction of high myopia. They are not commercially available from optical dispensaries, but rather are specially manufactured with instructions from the fitting practitioner, much like how a prosthetic is customized for an individual. The range of lens powers available to dispensing opticians for filling prescriptions, even in an aspheric form, is limited practically by the size of the image formed on the retina. High minus lenses cause an image so small that shape and form aren't discernible, generally at about −15 diopters, while high plus lenses cause a tunnel of imagery so large that objects appear to pop in and out of a reduced field of view, generally at about +15 diopters.

In prescriptions for both farsightedness and nearsightedness, the lens curve flattens toward the edge of the glass, except for progressive reading adds for presbyopia, where seamless vari-focal portions change toward a progressively more plus diopter. High minus aspheres for myopes do not necessarily need progressive add portions, because the design of the lens curvature already progresses toward a less-minus/more-plus dioptric power from the center of the lens to the edge. High plus aspheres for hyperopes progress toward less-plus at the periphery. The aspheric curvature on high plus lenses are ground on the anterior side of the lens, whereas the aspheric curvature of high minus lenses are ground onto the posterior side of the lens. Progressive add reading portions for plus lenses are also ground onto the anterior surface of the lens. The blended curvature of aspheres reduces scotoma, a ringed blind spot.

An intraocular lens (IOL) is an implanted lens in the eye, usually replacing the existing crystalline lens because it has been clouded over by a cataract, or as a form of refractive surgery to change the eye's optical power. The whole device usually comprises a small plastic lens with plastic side struts, called haptics, to hold the lens in place within the capsular bag inside the eye. IOLs were traditionally made of an inflexible material (e.g. PMMA) though this largely been superseded by the use of flexible materials. Most IOLs fitted today are fixed monofocal lenses matched to distance vision. However, other types are available, such as multifocal IOLs which provide the patient with multiple-focused vision at far and reading distance, and adaptive IOLs which provide the patient with limited visual accommodation.

Intraocular lenses have been used since 1999 for correcting larger errors in myopic (near-sighted), hyperopic (far-sighted), and astigmatic eyes. This type of IOL is also called PIOL (phakic intraocular lens), and the crystalline lens is not removed. More commonly, aphakic IOLS (that is, not PIOLs) are implanted via Clear Lens Extraction and Replacement (CLEAR) surgery. During CLEAR, the crystalline lens is extracted and an IOL replaces it in a process that is very similar to cataract surgery: both involve lens replacement, local anesthesia, are very quick (performed in about 30 minutes), and both require a small incision in the eye for lens insertion. Patients recover from CLEAR surgery quickly, typically within a week after surgery. During recovery, patients should avoid any activity that significantly elevates blood pressure. Patients should also be routinely monitored by their ophthalmologists. CLEAR has about a 90% success rate (risks include wound leakage, infection, inflammation, and astigmatism). CLEAR is typically performed on patients ages 40 and older to ensure that eye growth, which disrupts IOL lenses, will not occur post-procedure.

Once implanted, IOL lenses have three major benefits. First, they can be alternative to LASIK, a form of eye surgery that does not work for patients with serious vision problems. Effective IOL implants may also entirely eliminate the need for glasses or contact lenses post-surgery. Cataract will not return, as the lens has been removed. The disadvantage is that the eye's ability to change focus (that is accommodate) may have been reduced or eliminated, depending on the kind of lens implanted.

Special types of Phakic IOLs (PIOLs) are available in patients requiring IOL implantation without removal of crystalline human lens, particularly useful in refractive surgery for high myopia. For this, the eye surgeon has to determine the size of the PIOL. If the lens is of incorrect length, then it can rotate inside the eye, causing astigmatism, and/or damage to the natural lens. It can also block the natural flow of fluid inside the eye, causing glaucoma. The size is usually estimated, by measuring white-to-white, and estimating the ciliary sulcus diameter. However, the surgeon can perform 3D ultrasound biomicroscopy with for example Artemis for a completely accurate measurement. 3D ultrasound is to traditional 2D ultrasound as computer assisted tomography is to x-ray. Therefore, 3D ultrasound examination is strongly recommended, since the white-to-white guesstimate does not have a strong correlation with sulcus-to-sulcus—neither for myopic, nor for hyperopic eyes. About 1% of sulcus-to-sulcus estimates based on white-to-white are so wrong that serious complications can arise. This type of phakic lens has to be ordered from the manufacturer; requiring a number of weeks before the surgery. However, the routine posterior chamber IOLs (PC-IOLs) used for routine cataract surgical cases are available with the surgical suite or doctor's office, and the cataract surgery can usually be performed without delay once the patient is cleared for surgery. Recent surgical findings indicate that aphakic IOLs should also be measured carefully, as outsized IOLs can dislocate within the lens envelope, thus requiring either corrective surgery or aphakic lens removal and replacement.

Phakic IOLS (PIOLs) can be either spheric or toric—the latter is used for astigmatic eyes. The difference is that toric PIOLs have to be inserted in a specific angle, or the astigmatism will not be fully corrected, or it can even get worse.

According to placement site in the eyes phakic IOLs can be divided to:
Angle supported PIOLs: those IOLs are placed in the anterior chamber. They are notorious for their negative impact on the corneal endothelial lining, which is vital for maintaining a healthy dry cornea.
Iris supported PIOLs: this type is gaining more and more popularity. The IOL is attached by claws to the mid peripheral iris by a technique called enclavation. It is believed to have a lesser effect on corneal endothelium, though the iris is naturally delicate thus enclavation can cause eiris deterioration over time.
Sulcus supported PIOLs: these IOLS are placed in the posterior chamber in front of the natural crystalline lens. They have special vaulting so as not to be in contact with the normal lens. The main complications with this type is their tendency to cause cataracts and/or pigment dispersion.

Insertion of an intraocular lens for the treatment of cataracts is the most commonly performed eye surgical procedure. The procedure can be done under local anesthesia with the patient awake throughout the operation. The use of a flexible IOL enables the lens to be rolled or folded for insertion into the capsule through a very small incision, thus avoiding the need for stitches, and this procedure usually takes less than 30 minutes in the hands of an experienced ophthalmologist. The recovery period is about 2-3 weeks and, again, patients should avoid strenuous exercise or any activity that significantly increases blood pressure. Patients should also schedule regular visits with their ophthalmologists for several months so as to monitor the implants.

IOL implantation carries several risks associated with eye surgeries, such as infection, loosening of the lens, lens rotation, inflammation, night-time halos. Although IOLs enable some patients to have reduced dependence on glasses, most patients still rely on glasses for driving and reading.

While significant advances have been made in the optical quality of aphakic lenses, most lenses currently made have an overall optical thickness of one millimeter or greater at the center optical focal point (e.g. see U.S. Pat. No. 4,363,142). In the late 1990's, two patents were applied for and subsequently issued for lens optics significantly thinner than the afore-referenced lens patents (U.S. Pat. Nos. 6,096,077 and 6,224,628). Although improved, the extreme thinness of the lens manufactured in accordance with U.S. Pat. No. 6,096,077 caused some minor distortions of the optic once in the eye, while the lens manufactured in accordance with U.S. Pat. No. 6,224,628 was poured of molded silicone and did not provide the desired visual acuity.

### SUMMARY OF THE INVENTION

The present invention overcomes the problems and disadvantages associated with current strategies and designs and provides new intraoptical lens designs as well as methods for their manufacture and use.

One embodiment of the invention is directed to an intraocular lens that has a maximum thickness of 500 microns and is implantable into a mammalian eye. Preferably the mammalian eye is a human eye, but it may be another animal. Preferably the posterior surface of the lens has an asphericity correction. The lens is preferably composed of an acrylic, polymethylmethacrylate or silicone compound, or a combination thereof. Preferred lenses are from 18-26% hydrophilic, or from 74-82% hydrophobic, with a preferred optic diameter of less than, greater than, or equal to 6 millimeters. The lens optic diameter and the center thickness may be obtained by placing concentric rings on the anterior surface of the lens away from the natural lens capsule, by placing concentric rings on the posterior surface of the lens contacting the natural lens capsule, or by both. Preferably, the concentric rings are concave, convex or piano wherein each comprise a step that provides a change in thickness. Also preferably, the angle of an edge of the step that increases or decreases thickness is equal to the angle at which light rays traverse a surface of said step. The steps are preferably designed such that light rays that traverse the surface of said step converge on a single focal point of a retina when implanted into the lens envelope of a mammalian eye. Preferably, each step that provides a change in thickness to the lens is approximately ten microns, or the change may be more or less than 10 microns.

Another embodiment of the invention is directed to an intraoptical lens that has concentric rings on both sides. In this lens, light rays that contact a step between the concentric ring surface of one side of the lens also preferably contact a step between the concentric ring surface of the other side of the lens.

Another embodiment of the invention is directed to an intraocular lens that provides up to 45 diopters of power for vision correction. Preferably, lenses of the invention have a diopter flexibility at 0.25 diopter increments. Preferably, the surface of the lens contacting the natural lens capsule is optically concave and physically approximately piano. Alternatively, the surface contacting the natural lens capsule may be optically convex and physically approximately piano. Each concentric ring preferably has a radius that is corrected to allow light rays to focus on the retina to allow for distant vision. Also, forward movement of the lens allows for near vision.

Another embodiment of the invention is directed to methods of correcting vision comprising the lens of the invention. The original or natural lens crystal is removed surgically and the new optical lens inserted into the lens envelope. Alternatively, the optical lens may be inserted in addition to the natural lens crystal.

Another embodiment of the invention is directed to method of manufacturing an intraocular lens of the invention. Preferably, the intraocular lens possesses one or more concentric rings on an anterior or posterior surface, wherein each concentric ring is formed by developing the lens until a minimum or maximum thickness is obtained, then increasing or decreasing the thickness.

Other embodiments and advantages of the invention are set forth in part in the description, which follows, and in part, may be obvious from this description, or may be learned from the practice of the invention.

### DESCRIPTION OF THE DRAWINGS

FIG. 1. Anterior surface of optical lens showing multiple concentric rings.

FIG. 2. Transverse view of the optical lens showing anterior concentric circles.

FIG. 3. Magnification of lens showing stepped structure of anterior concentric rings.

FIG. 4. Magnification of lens showing stepped structure of posterior concentric rings.

FIG. 5. Sketch showing magnification of concentric rings on anterior and posterior surfaces.

FIG. 6. Sketch showing position of lens relative to structures of the human eye.

### DESCRIPTION OF THE INVENTION

An intraocular lens (IOL) is an implanted lens in the eye, usually replacing the existing crystalline lens that has been clouded over by a cataract, or as a form of refractive surgery to change the eye's optical power. Intraocular lenses can correct errors in myopic (near-sighted), hyperopic (far-sighted), and astigmatic eyes. The basic lens is comprised of a small plastic lens with plastic side struts, called haptics, to hold the lens in place within the capsular bag inside the eye. IOLs were traditionally made of an inflexible material (such as for example PMMA) though this largely been superseded by the use of flexible materials. Most IOLs fitted today are fixed monofocal lenses matched to distance vision, although multifocal IOLs are available which provide the patient with multiple-focused vision at far and reading distance, and also adaptive IOLs which provide the patient with limited visual accommodation. While significant advances have been made in the optical quality of lenses, most lenses have an overall optical thickness of one millimeter or greater at the center optical focal point.

It has been surprisingly discovered that intraocular lens can be manufactured that are substantially thinner and provide improved comfort and greater visual acuity to the patient. Thinner lenses provide reduced volume and weight, and thereby less pressure to the eye, and greater clarity of vision, and there is less light refraction and reflection within the lens itself. Thinner lens allow for increased light penetration (that is an enhanced optical acuity) and a wider range of vision corrections. Further, these lenses offer greater stability within the eye than the conventional intraocular lenses as the extreme thinness of the lens allows it to he implanted through a small incision, which has been demonstrated to produce fewer post-surgical complications, such as inflammation or infection. Another advantage is that these lenses preferably provide 10 micron step increments to create a more gradual progression of lens thickness, center to edge, thus providing a more continuous and smoother optical effect to the eye. The steps in the lens may be convex, concave, or piano, depending upon the optical measurement of each eye, providing therefore customized diopter strength to each eye, whether naturally myopic, hyperopic or emmetropic. The inventive lens can be manufactured with precision to diopters strengths of up to 28 diopters, in ¼ diopters increments. In each case, the angle of the edge of each step is defined as the angle at which light would be bent passing through that point in the lens to reach the focal point on the retina for distance vision.

Other embodiments of the inventive lens provide for concentric rings on both anterior and posterior surfaces thus potentially increasing lens power up to 45 diopters while maintaining maximum lens thickness of less than 500 micrometers.

The lens offers greater stability within the eye than the ultra-thin lenses, while the 10 micron steps preferred create a gradual progression of lens thickness, center to edge, thus providing a continuous and smooth optical effect to the eye. The lens is preferably deformable and can be constructed of a variety of acrylic or silicone substances known to be benign in the eye, and which may have hydrophilic or hydrophobic properties, varying elastic strengths and optical clarity qualities. The preferred material for the lens is 18% hydrophilic acrylic material, selected in this case for its tensile strength and superior optical acuity in light transfer. The lens is preferably designed for insertion into the eye through the cornea, and lodged by means of attached haptic, in the lens envelope once the natural crystalline lens has been surgically removed. The attachment location for the lens haptic is in the equator of the lens envelope, and the lens posterior surface is contiguous with the posterior of the lens envelope.

In other embodiments, the lens haptic may be attached to the lens capsule at a point or points other than the capsular equator. In other embodiments the lens haptic may be fixated at the ciliary sulcus. In other embodiments the lens haptic may be fixated at the angle of the anterior chamber of the eye.

The lens is preferably manufactured to a predetermined maximum material thickness of 500 μm or less and preferably 475 μm or less, more preferably 450 μm or less, more preferably 425 82 m or less, more preferably 400 μm or less or more preferably 375 μm or less. A minimum material thickness is predetermined to allow the lens to be folded, or otherwise compressed without permanent creasing, into a particular syringe for insertion in the eye with an insertion device (e.g. IOL-specific injector). Folding is preferred over rolling when inserting the lens, in part, because rolling can increase the curve of the lens material, and unrolling in the eye can cause the rolled lens to brush up against the natural eye tissue, potentially causing trauma.

The inventive lens has an anterior concave surface comprised of a series of concentric annular rings that may be concave, convex, or planar in order to provide specific vision strength to the patient. The steps formed by these annular rings are preferably approximately 10 μm high in each instance, and may have varying widths as low as preferably 7 μm so as to provide a contiguous optical surface to the patient, while enhancing the precision of light focus on the retina. The increase in step width is consistent with the development of the steps to the main focal point of the lens, which is to be placed directly behind the center focal point of the iris. The lens center may be aspherical, such asphericity designed for maximum diopter efficiency and light transfer.

As an alternative design application, the lens may be manufactured with an anterior convex surface either smooth and aspherical or comprised of a series of concentric annular rings that may be convex, concave or planar.

The lens preferably possesses a series of posterior surfaces in accordance with the specific needs of the patient. In the first instance the posterior surface may be an aspheric, smooth surface with asphericity specified to tune the concave or convex or planar anterior surface steps so as to refract light to a precise focal point on the retina for distance vision. A preferred maximum thickness of the lens as measured between the apex of the aspherical posterior surface and the apex of the center of the anterior stepped surface is to be less than 475 μm. The minimum thickness of the lens at the periphery of the central anterior surface is to be at or above the minimum thickness required to allow the lens material to retain its pre-flexed shape subsequent to flexing.

In the second instance, the posterior surface of the lens may have a series of concave, convex or planar steps in the form of concentric annular rings that may give the posterior of the lens the appearance of a flat surface. In this instance, the placement and configuration of the posterior rings will be such as to be tuned to the placement of the anterior annular rings, thereby providing an increase in the power of the lens without requiring additional thickness. In all cases the edges of the concentric rings may be angled so as to be equal to the angle of light passing through the lens at any such point, thereby eliminating any perception of the rings by the patient. If both anterior and posterior annular rings are utilized, the lens can be manufactured to up to 10, 20, 30, 40 or 45 diopters of power (or any power in between), while maintaining the maximum thickness of 500 μm or less.

In the third instance, the posterior surface of the lens may be contoured with a toric adjustment to correct astigmatism. In such an instance, the inventive lens will have a specific orientation mark on the haptic, to assist the implanting ophthalmologist in properly orienting the lens in the patient's eye.

The radial width of the central disc of the anterior surface will be determined by the asphericity, convexity or concavity assigned to the anterior surface and the thickness of that surface as necessary to give the lens its desired optical power. Similarly, the radial width of each annular ring will be determined by the required power of the lens, and the thickness of such ring at its thickest point, though in no case should the thickness of any ring be greater than the maximum thickness of the lens. Similarly, the minimum thickness of the juncture between each annular ring and the face of the next outward ring should be greater than or equal to the predetermined minimum thickness.

The outer perimeter of the inventive lens has a surface tangent to the curvature of the natural lens capsule's posterior surface. In this configuration, the inventive lens should mitigate the development of Posterior Capsule Opacification (PCO) directly behind the lens optic. The inventive lens, when measured directly across the diameter from outer perimeter point to opposing outer perimeter point, will have measurement of 6 millimeters (mm), though in certain instances the lens may measure greater than 6 mm in diameter, and in other instances the lens may measure less than 6 mm, depending upon the particular needs and eye geometry of the patient.

An alternative placement option, commensurate with appropriate haptics, allows the lens to be placed outside of the natural lens capsule, directly behind the iris, and with haptic fixation in the ciliary sulcus. The lens power in this instance is calculated to provide suitable diopters strength for distance vision given the new position of the lens in the eye. Focal flexibility is obtained through the haptic design responsive to movement of the ciliary body. In certain cases, for higher diopters requirements, the overall lens maximum thickness may be increased to greater than 475 microns.

The following examples illustrate embodiments of the invention, but should not be viewed as limiting the scope of the invention.

### EXAMPLES

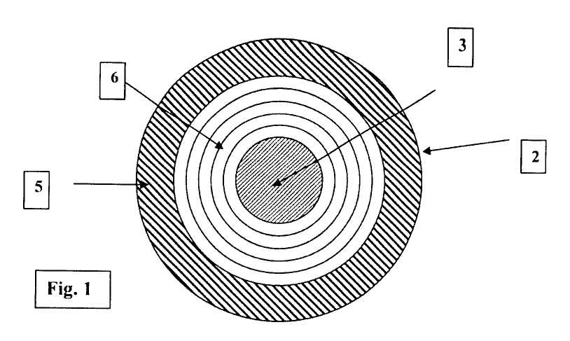

FIG. 1 is a sketch depicting the anterior surface of an intraoptic lens. Depicted is the optical edge of the lens (optical diameter edge to edge or outer optical perimeter) (2), with a lens center thickness, in this case at 475 microns measured at maximum depth at center of optical focal point (3). Shown is also the ring height or maximum thickness of the lens in the central optical zome (4) and rings on anterior surface at peripheral optical area showing the concentric stepped rings for distance vision (5).

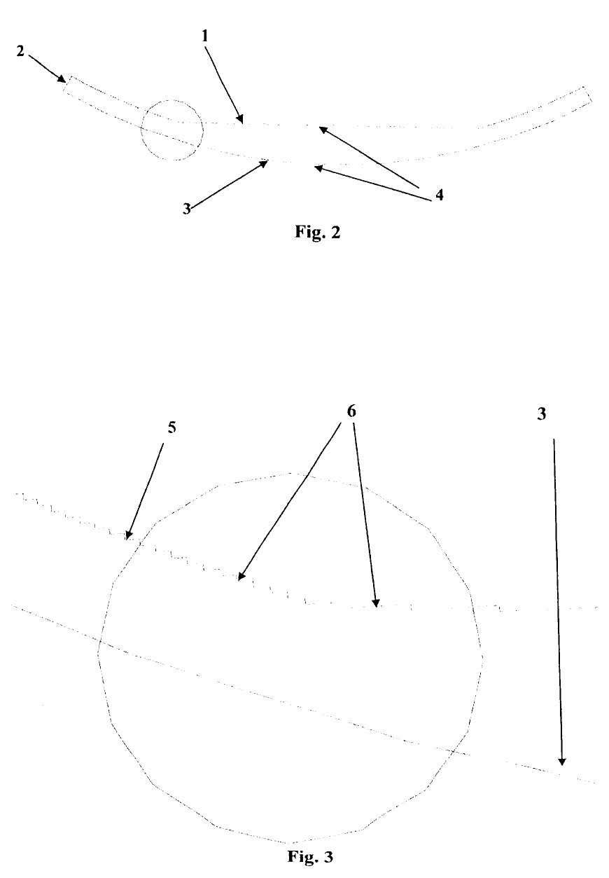

FIG. 2 is a sketch depicting the anterior surface (1), lens thickness or outer perimeter of the optic (2), and center optical focal point (3). Ring height (4), is where concentric rings appear.

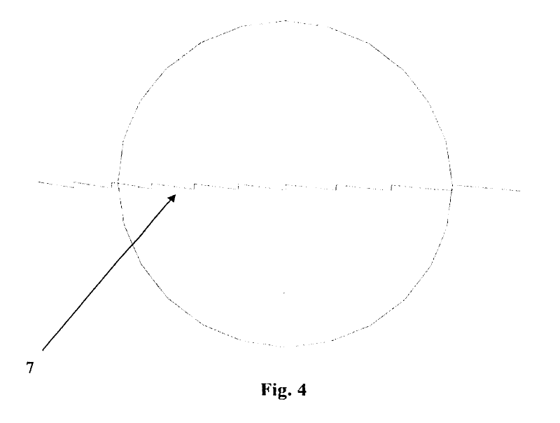

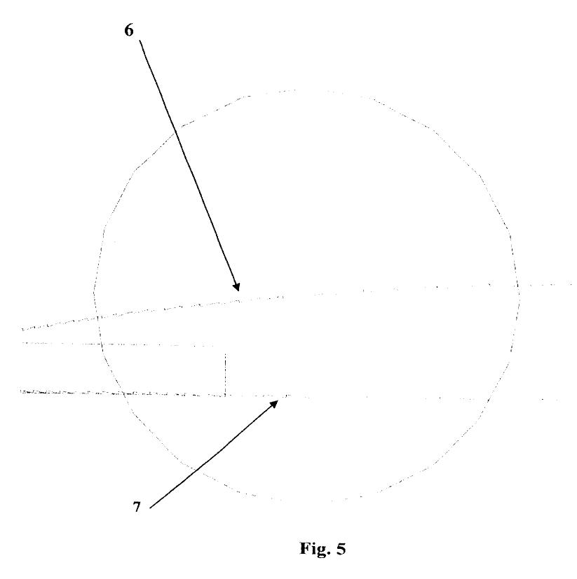

FIG. 3 depicts a magnification of the circular area of FIG. 2, showing the stepped ring surface structure at the anterior of the lens (5) and at center optic area of the lens (intermediate band of the lens showing concentric stepped rings) (6). Lens thickness (3) is measure at the maximum depth at center of lens optical focal point. FIG. 4 depicts a magnification of concentric rings (7) on posterior surface at central optical area.

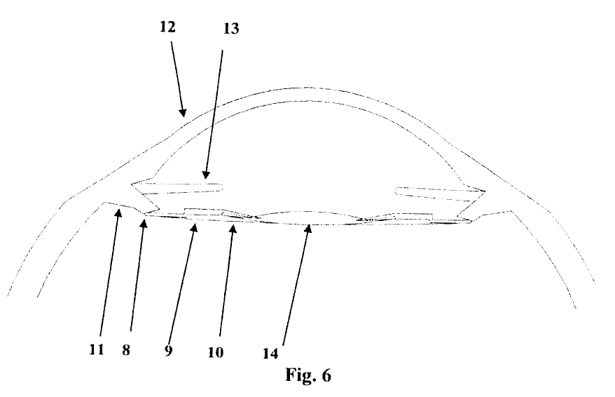

FIG. 5 depicts the rings on the anterior surface (6) and the posterior surface (7) of the optical lens.

FIG. 6 is a sketch depicting the placement of the optical lens relative to structures of the human eye. The tip of the lens haptic (position of lens haptic at capsular equator) (8) is shown as it rests against the equator of the capsule is held in position by the zonules (central arm of haptic) (9). Zonules are the hair-like structures that attach to the natural lens and the ciliary body and hold the natural lens in position. Zonules aide to change the shape of the natural lens for near vision correction. The lens envelope (posterior capsule) (10) is where the natural lens is removed and the artificial lens inserted.

The ciliary body (11) of the eye is shown which changes shape to allow the natural lens to change shape to give the patient near vision. The cornea (12) is the clear portion of the eye that refracts (bends) light. Along with the natural lens the light is bent to come to focus on the retina. The iris (13) or the colored portion of the eye is used to meter the amount of light allowed into the eye. The intraocular lens (14) is shown as it would appear in the far (distance) position in the eye.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of Furthermore, the terms comprising, including, and containing are not intended to be limiting. It is intended that the specification and examples be considered exemplary only with the true scope and spirit of the invention indicated by the following claims.

1. An intraocular lens that has a maximum thickness of 500 microns and is implantable into a mammalian eye.

2. The lens of claim 1, wherein the posterior surface of the lens has an asphericity correction.

3. The lens of claim 1, which is from 18-26% hydrophilic.

4. The lens of claim 1, which is 74-82% hydrophobic.

5. The lens of claim 1, wherein the optic diameter is less than or equal to 6 millimeters.

6. The lens of claim 1, wherein the optic diameter is greater than or equal to 6 millimeters.

7. The lens of claim 6, wherein the optic diameter is less than or equal to 9 millimeters.

8. The lens of claim 1, wherein the optic diameter is greater than or equal to 9 millimeters.

9. The lens of claim 1, which is comprised of an acrylic compound.

10. The lens of claim 1, which is comprised of a polymethylmethacrylate compound.

11. The lens of claim 1, which is comprised of a silicone.

12. The lens of claim 1, wherein the optic diameter and the center thickness are obtained by placing concentric rings on the anterior surface of the lens away from the natural lens capsule, by placing concentric rings on the posterior surface of the lens contacting the natural lens capsule, or by both.

13. The lens of claim 12, wherein the concentric rings are concave, convex or piano.

14. The lens of claim 13, wherein the concentric rings each comprise a step that provides a change in thickness.

15. The lens of claim 14, wherein the angle of an edge of the step that increases or decreases thickness is equal to the angle at which light rays traverse a surface of said step.

16. The lens of claim 15, wherein the light rays that traverse the surface of said step converge on a single focal point of a retina when implanted into the lens envelope of a mammalian eye.

17. The lens of claim 14, wherein the step that provides a change in thickness is approximately ten microns.

18. The lens of claim 14, wherein the step that provides a change in thickness is greater than 10 microns.

19. The lens of claim 14, wherein the step that provides a change in thickness is less than 10 microns.

20. The lens of claim 12, which has concentric rings on both sides, wherein light rays contact a step between the concentric ring surface of one side of the lens also contact a step between the concentric ring surface of the other side of the lens.

21. The lens of claim 1, which provides up to 45 diopters of power for vision correction.

22. The lens of claim 1, which has a diopter flexibility at 0.25 diopter increments

23. The lens of claim 1, wherein the surface contacting the natural lens capsule is optically concave and physically approximately piano.

24. The lens of claim 1, wherein the surface contacting the natural lens capsule is optically convex and physically approximately piano.

25. The lens of claim 1, where each concentric ring has a radius that is corrected to allow light rays to focus on the retina to allow for distant vision.

26. The lens of claim 1, wherein forward movement of the lens allows for near vision.

27. The lens of claim 1, wherein the mammalian eye is a human eye.

28. A method of correcting vision comprising:
a. surgically removing a natural lens of an eye of a patient; and
b. inserting the lens of claim 1 into the eye.

29. The method of manufacturing an intraocular lens that possesses one or more concentric rings on an anterior or posterior surface, wherein each concentric ring is formed by developing the lens until a minimum or maximum thickness is obtained, then increasing or decreasing the thickness.

30. The method of claim 29, wherein the intraocular lens has an optic diameter of greater than or equal to 6 millimeters.

31. The method of claim 30, wherein the optic diameter is less than or equal to 9 millimeters.

32. The method of claim 29, wherein the intraocular lens has an optic diameter of greater than or equal to 9 millimeters.
